(19) United States
(12) Patent Application Publication
(10) Pub. No.: US 2014/0054189 A1
(43) Pub. Date: Feb. 27, 2014

(54) NASAL FLUSHING DEVICE

(71) Applicants: Lih-Chiu Wu, Kaohsiung City (TW); Hui-Shuan Lee, Kaohsiung City (TW)

(72) Inventors: Lih-Chiu Wu, Kaohsiung City (TW); Hui-Shuan Lee, Kaohsiung City (TW)

(21) Appl. No.: 13/592,919

(22) Filed: Aug. 23, 2012

(51) Int. Cl. A61M 3/02; A61B 19/02

(52) U.S. Cl. 206/370; 604/239

(57) ABSTRACT

A nasal flushing device includes a catheter unit, a connecting unit, and an injection unit. The catheter unit includes a catheter body, and the plural of openings formed in the body of the catheter, the catheter body has an open end and a closed end. The connection unit includes a connect part and a combination part. The end of the set of the connect part connecting pipe connected with the open end, opposite the other end of the combination part connection. The injection unit including the combination part syringe connected together, and a putter set in the syringe.

### BACKGROUND OF THE INVENTION

1. Field of the Invention

The present invention relates to a nasal flushing device, and more particularly to a portable nasal flushing device.

2. Description of the Related Art

Recently, with climate change and over development, air population becomes a serious issue in our daily life. According to Medscape website (http://emedicine.medscape.com/article/232791-overview#a0156) in USA every 1000 people, there are 146 people have chronic nasosinusitis, and roughly estimation there are about 18 to 22 million doctor visits and 3.4 to 5.0 billion direct medical spent. However, the numbers still increase every year. So far, according to Medscape and Mayo Clinic websites, there is no cure for chronic nasosinusitis. The main symptoms are nasal obstruction, post nasal drip and cough. Surgical solution is only suitable for some cases, and antibiotics are effective for acute bacterial infection rather than chronic conditions. The only recommendation treatment for patients have chronic symptoms to keep the nasal cavity moisten all time as home treatment.

Please refer to FIG. 1. In the nasal cavity between the nasal vestibule and nasopharynx is divided by the superior turbinate A, the middle turbinate B, the inferior turbinate C, the upper nasal meatus A1, the middle nasal meatus B1 and the lower nasal meatus C1. All secretion from the paranasal sinuses is exited from the several openings located in the upper nasal meatus A1 and the middle nasal meatus. Therefore, thick tenacious secretion generated by chronic nasosinusitis mainly builds up the upper nasal meatus and the middle nasal meatus nasal congestion, which causes nasal obstruction.

In order to moisturize the nasal cavity, the options are warm damp facial cover, warm steam inhalation and nasal irrigations such as neti pot can be applied. However, the first two solutions cannot bring the moisture directly into the nasal cavity.

Furthermore, most of improvements for the nasal irrigation devices in the market are related to supplying mechanisms instead of changing water flow direction.

Please refer to FIG. 2. As Taiwan patent No. M418689 discloses a nasal irrigation device has: an applying portion 10 and a nozzle 20. The applying portion 10 has an inner space 11 and the nozzle 20 has an opening 21 and an extending tube 22 reaching towards to the inner space 11 of the applying portion 10. When the inner space 11 is filled with water and compressed to push the water into the extending tube 22 and exit from the opening 21.

As previous description, nasosinusitis patient needs to moist the upper nasal meatus A1 and the middle nasal meatus B1, the above mentioned nasal irrigation device can bring water to the nasal vestibule. However, due to the dimension of the nozzle 20, it can only provide a single direction spout at the nasal vestibule, which can just reach to the lower nasal meatus not deep into the upper nasal meatus A1 and the middle nasal meatus B1 to treat the symptoms correctly.

Moreover, the spout provided by the device might be too strong for nasal and nasopharyngeal mucosa or choke the patient.

Therefore, it is desirable to provide a nasal flushing device to mitigate and/or obviate the aforementioned problems.

### SUMMARY OF THE INVENTION

An objective of the present invention is to provide an easy nasal flushing device.

In order to achieve the above mentioned objective, the nasal flushing device includes a catheter unit and an injection unit.

The catheter unit has a catheter body and a plurality of apertures formed on the catheter body, the catheter body having an open end and a closed end opposite the open end. The injection unit has a detachable syringe and a putter set provided in the syringe.

The nasal flushing device utilizes the catheter unit to deeply rinse the nasal cavity, and the combination of the catheter unit, the connecting unit, the injection unit, and the travel kit provides portability to the user.

Other objects, advantages, and novel features of the invention will become more apparent from the following detailed description when taken in conjunction with the accompanying drawings.

### BRIEF DESCRIPTION OF THE DRAWINGS

FIG. 1 is anatomy cross-sectional view of the nasal cavity.

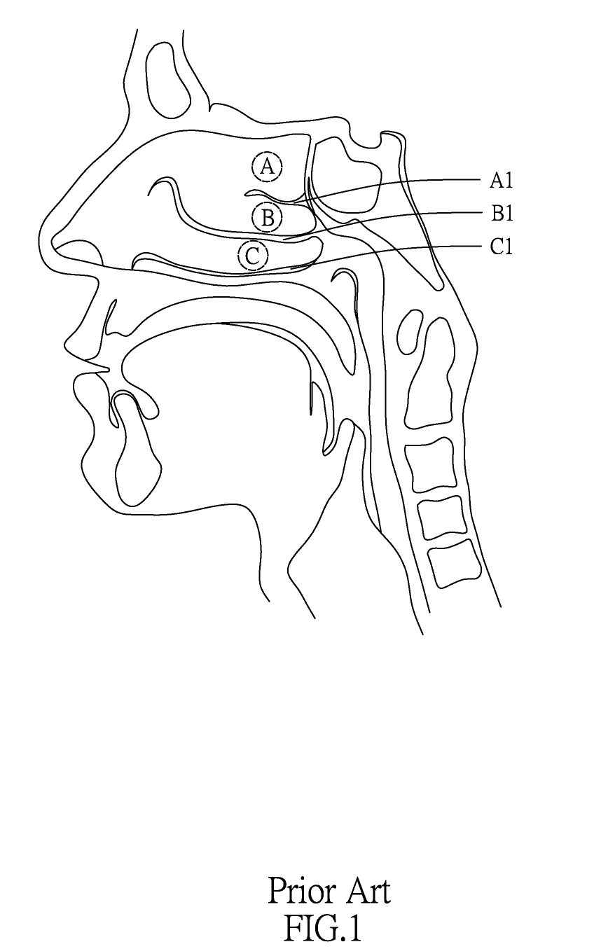

FIG. 2 is a cross-sectional view of a nasal irrigation device disclosed in Taiwan patent No. M418689.

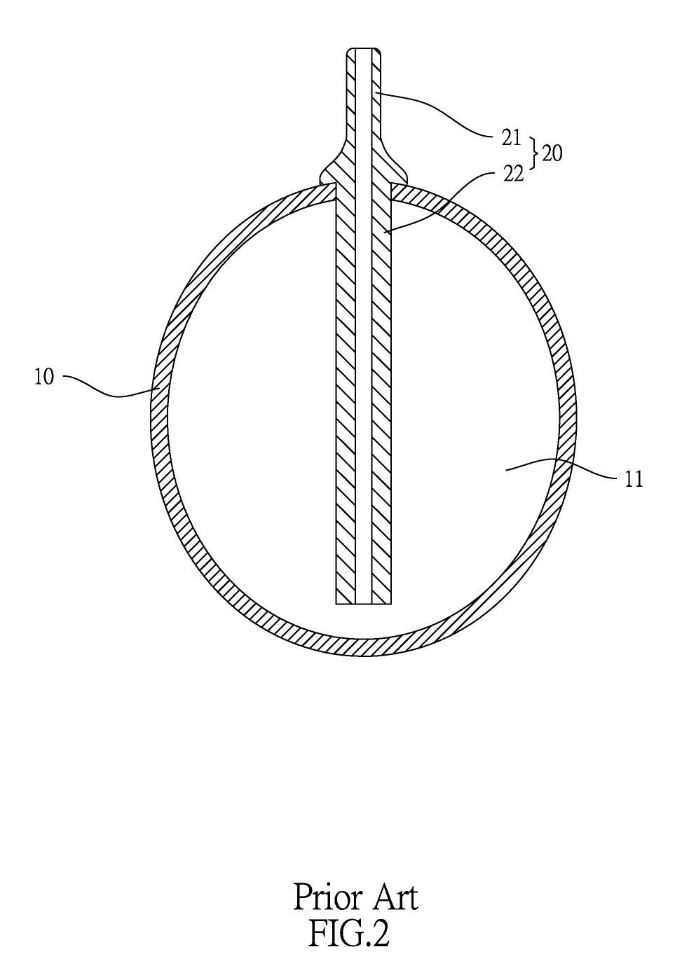

FIG. 3 is a perspective view of a nasal flushing device according to a first embodiment of the present invention.

FIG. 4 is a schematic drawing showing the nasal flushing device of the first embodiment being applied to the nasal cavity.

FIG. 5 is another schematic drawing showing the nasal flushing device of the first embodiment being applied to the nasal cavity.

FIG. 6 is a perspective view of the nasal flushing device according to a second embodiment of the present invention.

FIG. 7 is a perspective view of the nasal flushing device according to a third embodiment of the present invention.

### DETAILED DESCRIPTION OF THE PREFERRED EMBODIMENT

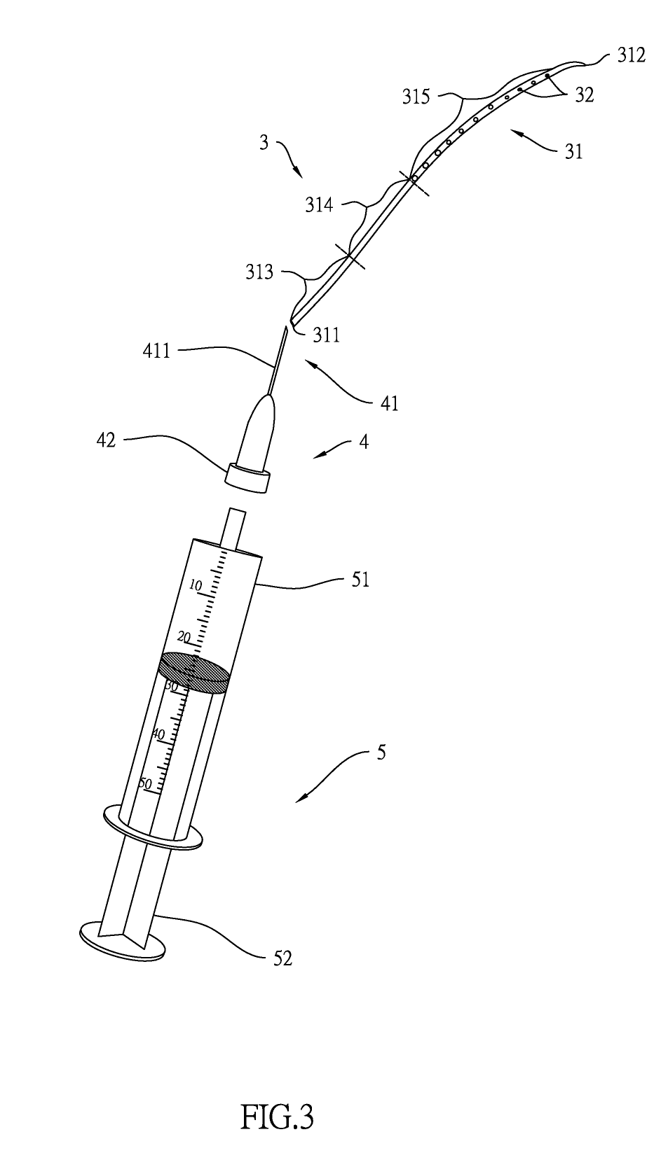

Please refer to FIG. 3. FIG. 3 is a perspective view of a nasal flushing device according to a first embodiment of the present invention. The nasal flushing device comprises a catheter unit 3, a connecting unit 4, and an injection unit 5.

The catheter unit 3 has a catheter body 31 and a plurality of apertures 32 formed on the catheter body 31. The catheter body 31 has an open end 311 and a closed end 312 opposite the open end 311, and the closed end 312 curved such that the catheter body 31 is capable of entering deeply into the nasal meatus.

The catheter body 31 from the open end 311 to the closed end 312 is divided into a connecting section 313, a reaching section 314 and a flushing section 315. The connecting section 313 has a length from 1 to 5 cm, the reaching section 314 has a length from 1 to 5 cm, and the flushing section 315 has a length from 3 to 10 cm. The plurality of apertures 32 on the catheter body 31 are disposed on the flushing section body 315, and wherein the apertures 32 further far away from the closed end 312 have larger inner diameters than the apertures 32 closer to the closed end 312. Furthermore, the catheter body 31 is made of silicon or latex such soft material, which can easily be bent to enter into the curved nasal meatus. Since average nasal meatus of patient with chronic sinusitis is not wider than 3 mm, a diameter of the catheter body 31 is not larger than 3 mm.

The connecting unit 4 has a connecting portion 41 and an assembling portion 42. One end of the connecting portion 41 is provided with a connecting tube 411 connected to the open end 311, and another end of the connecting portion is connected to the assembling portion 42.

In addition, since the diameter of the connecting tube 411 is smaller than the diameter of the catheter body 31 and it causes resistance to passing fluid, a length of the connecting tube 411 should be shorter to reduce the resistance.

The injection unit 5 has a detachable syringe 51 and a putter set 52 provided in the syringe 51. The syringe 51 and the putter set 52 are used for compressing the fluid in the container, to form injecting spout. There can be various combinations of the syringe 51 and the putter set 52 should not be limited by this embodiment.

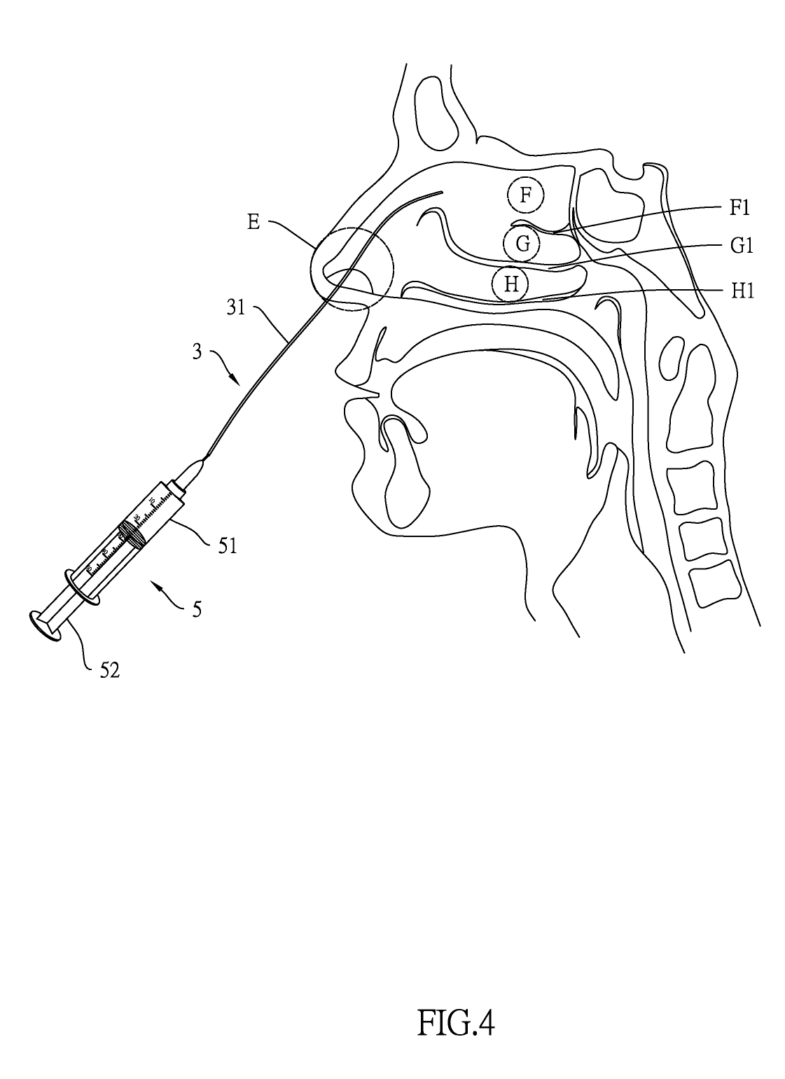

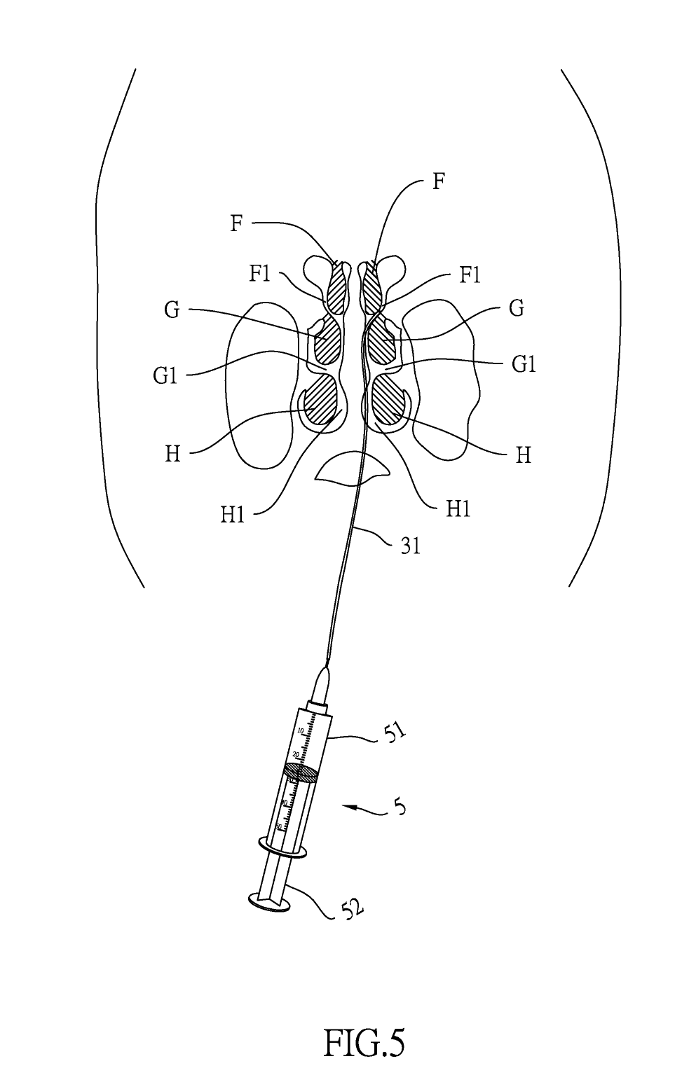

Please refer to FIG. 4 and FIG. 5 together. As showing the drawings, the nasal cavity has a nasal vestibule E, a superior turbinate F, a middle turbinate G, a inferior turbinate H, a upper nasal meatus F1, a middle nasal meatus G1 and a lower nasal meatus H1. The nasal vestibule usually is about 4 cm long, the upper nasal meatus F1, the middle nasal meatus G1 and the lower nasal meatus H1 are usually about 5 cm in length. Therefore, in the first embodiment, a length of the flushing section 315 of the catheter body 31 is 5 cm, the plurality of apertures 32 are arranged apart about every 0.5 cm to 1 cm and around the surface of the catheter body 31. A total length of the catheter body 31 is about 17 cm, the length of the flushing section 315 is 5 cm, the length of the reaching section 314 in the nasal vestibule E is 4 cm, the remaining length of the connecting section 313 is 8 cm, which can be held by the user and provide further reaching length.

For actual application, the user inserts the flushing section 315 of the catheter body 31 into the upper nasal meatus F1 or the middle nasal meatus G1, fills the syringe 51 with flushing fluid, then pushes the putter set 52 to push the flushing fluid in the syringe 51, such that the flushing fluid spurs out from the aperture 32 on the flushing section 315 of the catheter body 31. Therefore, multiple thin spouts spur out with injecting angles nearly perpendicular to the catheter body 31. With injecting angles nearly perpendicular to the catheter body 31, the catheter body 31 is more likely free from counterforce movement during the injection, and the flushing fluid can quickly fill up the nasal meatus.

Since the flushing section 315 is configured to be disposed in the upper nasal meatus F1 of the nasal cavity, the flushing fluid can directly rinse the mucus or crust built up in the upper nasal meatus F1. Similarly, when the flushing section 315 is configured to be disposed in the middle nasal meatus G1 or the lower nasal meatus H1 the flushing fluid can directly rinse the mucus or crust built up in the middle nasal meatus G1 and the lower nasal meatus H1.

Moreover, the catheter body 31 is made of silicon or latex material and has a small dimension, when the user insert the catheter body 31 into the nasal cavity, if he or she feels any discomfort, he or she can immediately stop to avoid damaging the tissue of the nasal cavity. Furthermore, if the catheter body 3 is not completely inserted into the upper nasal meatus F1, the spouts with injecting angles nearly perpendicular to the catheter body 31 injected from aperture 32 of the flushing section 315 are capable of rinsing most of the area. In addition, some spouts might reach up to a 30 cm distance, which can pass through some thin gap into deeper area for a better cleaning result. Even though, some spouts might reach up to a 30 cm distance, but the numerous number of spouts do not have high pressure to damage the mucosa.

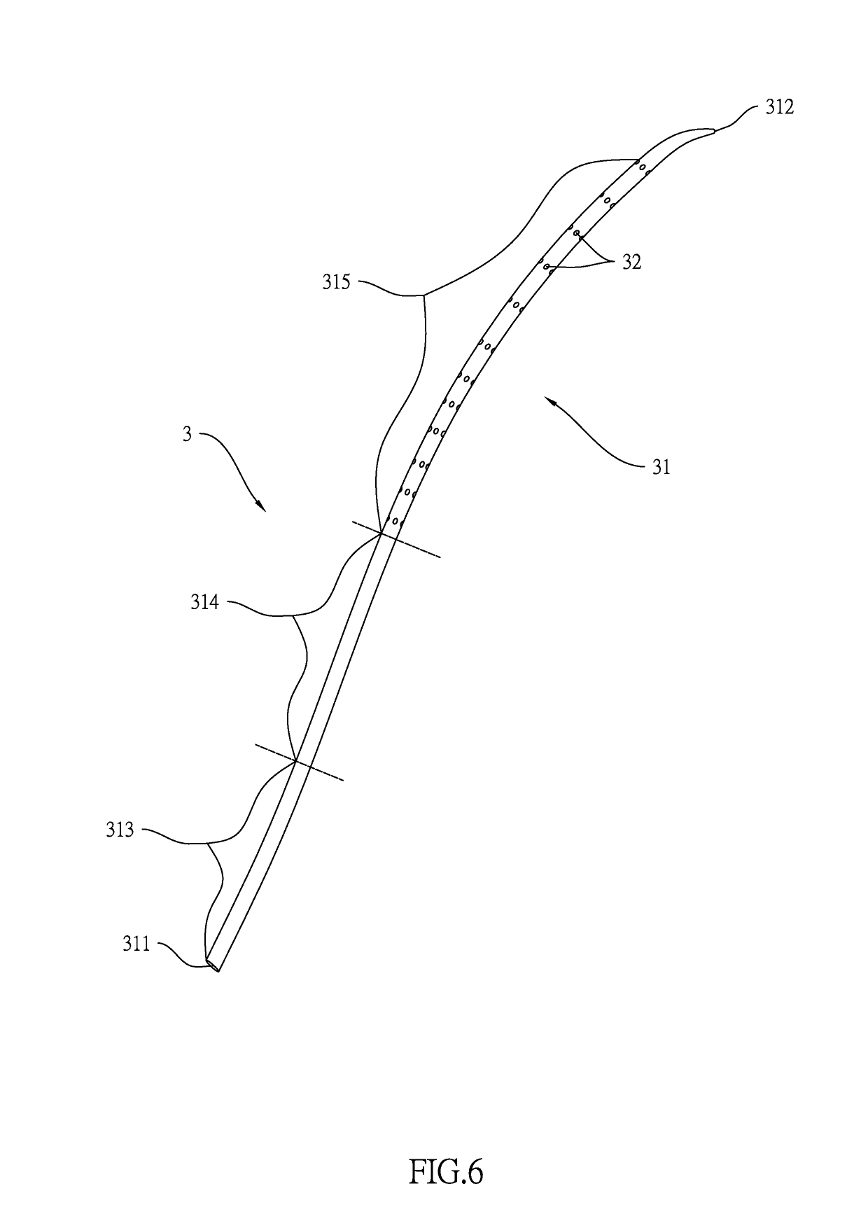

Please refer to FIG. 6. FIG. 6 is a perspective view of the nasal flushing device according to a second embodiment of the present invention. The second embodiment is substantially similar with the first embodiment, the difference is, every aperture 32 disposed on the flushing section 315 has an identical diameter, but the apertures 32 disposed further away from the closed end 312 on the flushing section 315 are arranged more closely together with respect to each other than the apertures disposed closer to the closed end 312.

Since the compressed spouts first release to lower pressure area, the spouts move towards to the closed end 312 and come out from the apertures. Because the apertures 32 disposed further away from the closed end are arranged more closely together with respect to each other than the apertures 32 disposed closer to the closed end 312, the entire flushing section 315 have relatively even flow and larger rinsing area.

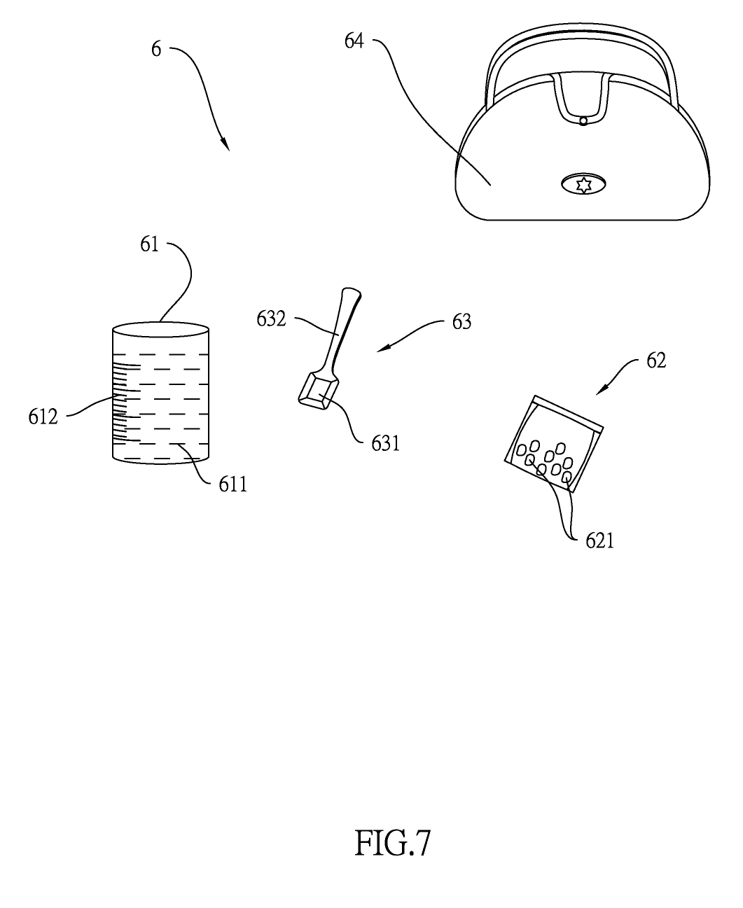

Please refer to FIG. 3 and FIG. 7. FIG. 7 is a perspective view of the nasal flushing device according to a third embodiment of the present invention. The third embodiment is substantially similar with the first embodiment, the difference is, the nasal flushing device further has a travel kit 6.

In the third embodiment, the travel kit 6 has a container 61 to provide fluid 611 for the injection unit 5, a salt package 62, a spoon 63, and a bag 64. The salt package has a plurality of salt crystals 621 for being dissolved in the fluid 611 in the container 61. The bag 64 is used for storing the catheter unit 3, the connecting unit 4, the injection unit 5, the container 61, salt package 62, and the spoon 63.

Moreover, the container 61 in the third embodiment is provided with a measurement scale 612, and the container 61 is capable of storing 220 c.c. fluid. The spoon 63 has a scoop portion 631 and a handle 632, and the scoop portion 631 is capable of holding 1 g salt crystals 621. Preferably, an inner dimension of the scoop portion 631 is about 0.8 cm×0.8 cm×0.72 cm=0.4608 cm³, and 0.4608 cm³×2.165 (salt specific weight)=1 gram.

Adding 1 gram salt crystals 621 into 100 c.c. water can make 0.9% normal saline most close to human body fluid. Therefore, with a correct amount of fluid in the container 61 and a correct amount of salt crystals 621, user can bring the travel kit 6 anywhere.

The user just needs to easily assemble the catheter unit 3, the connecting unit 4 and the injection unit 5 together, mix correct amount of salt crystals 621 and water 611 in the container 61 of the package 6, pull the mixed fluid into the syringe 51 of the injection unit 5, and then push the putter set 52 in the syringe 51 deeply into the nasal cavity.

Since the length of the flushing section 315 of the catheter body 31 is between 5 to 10 cm, and the apertures 32 on the flushing section 315 further far away from the closed end 312 have larger inner diameters than the apertures 32 closer to the closed end 312. The flushing fluid can rinse the upper nasal meatus F1 and the middle nasal meatus G1, and the plurality of apertures 32 with different diameter on the flushing section 315 can provide even pressure sprouts for more comfortable feeling to the user. After flushing, the user pulls the syringe 51 and the putter set 52 apart and stores the syringe 51, the putter set 52, the catheter unit 3 and the connecting unit 4 into the travel kit 6.

According to the above-mentioned description, the nasal flushing device utilizes the catheter unit 3 to deeply rinse the nasal cavity, and the combination of the catheter unit 3, the connecting unit 4, the injection unit 5, and the travel kit 6 provides portability to the user.

Although the present invention has been explained in relation to its preferred embodiment, it is to be understood that many other possible modifications and variations can be made without departing from the spirit and scope of the invention as hereinafter claimed.

What is claimed is:

1. A nasal flushing device comprising:
a catheter unit having a catheter body and a plurality of apertures formed on the catheter body, the catheter body having an open end and a closed end opposite the open end; and
an injection unit having a detachable syringe and a putter set provided in the syringe.

2. The nasal flushing device as claimed in claim 1 further comprising a connecting unit, the connecting unit having a connecting portion and an assembling portion, one end of the connecting portion provided with a connecting tube connected to the open end, and another end of the connecting portion connected to the assembling portion.

3. The nasal flushing device as claimed in claim 2, wherein the catheter body from the open end to the closed end is divided into a connecting section, a reaching section and a flushing section, the connecting section having a length from 1 to 5 cm, the reaching section having a length from 1 to 5 cm, and the flushing section having a length from 3 to 10 cm, and wherein the closed end is slightly curved.

4. The nasal flushing device as claimed in claim 3, wherein the plurality of apertures on the catheter body are disposed on the flushing section.

5. The nasal flushing device as claimed in claim 4, wherein the apertures further far away from the closed end have larger diameters than the apertures closer to the closed end.

6. The nasal flushing device as claimed in claim 5, wherein the flushing section is configured to be disposed in a nasal meatus, the reaching section is configured to be disposed in a nasal vestibule, and the connecting section is configured to be exposed outside of a nostril.

7. The nasal flushing device as claimed in claim 6 further comprising a travel kit, the travel kit having a container to provide fluid for the injection unit, a salt package and a spoon; wherein the salt package has a plurality of salt crystals for being dissolved in the fluid in the container.

8. The nasal flushing device as claimed in claim 7, wherein a measurement scale is provided on a surface of the container, the spoon having a scoop portion and a handle, and the scoop portion is capable of holding one gram salt.

9. The nasal flushing device as claimed in claim 8, wherein the travel kit further includes a bag for storing the container, the spoon and the salt package.

10. The nasal flushing device as claimed in claim 3, wherein the plurality of apertures disposed further away from the closed end on the flushing section are arranged more closely together with respect to each other than the apertures disposed closer to the closed end.
